(19) United States
(12) Patent Application Publication
(10) Pub. No.: US 2025/0376438 A1
(43) Pub. Date: Dec. 11, 2025

(54) METHOD FOR PRODUCING ISOCYANATES VIA PHOSGENATION OF A MIXTURE OF (AR)ALIPHATIC DIAMINES AND CYCLOALIPHATIC DIAMINES

(71) Applicant: Covestro Deutschland AG, Leverkusen (DE)

(72) Inventors: Frank Richter, Leverkusen (DE); Tim Loddenkemper, Dormagen (DE)

(21) Appl. No.: 18/876,701

(22) PCT Filed: Jun. 20, 2023
(86) PCT No.: PCT/EP2023/066614

§ 371 Date: Dec. 19, 2024

(30) Foreign Application Priority Data
Jun. 22, 2022 (EP) 22180309.1
Jun. 22, 2022 (EP) 22180310.9
Jan. 5, 2023 (EP) 23150427.5

(51) Int. Cl. C07C 263/10

(52) U.S. Cl. CPC C07C 263/10

(57) ABSTRACT

The invention relates to a method for producing isocyanates via a gas-phase phosgenation of the corresponding amines, wherein an amine mixture formed of at least one first amine and at least one second amine, that is different from the first amine, is reacted with phosgene to produce a reaction mixture, wherein the method is characterized in that the first amine is selected from the group comprising or consisting of aliphatic and/or araliphatic amines and the second amine is selected from the group comprising or consisting of cycloaliphatic amines. The invention also relates to a product that is/can be obtained using this method and to the use of a product of this type. The invention also relates to the use of an amine mixture formed of at least one first amine and at least one second amine, that is different from the first amine, in a method for producing the corresponding isocyanates via gas-phase phosgenation in order to lower the content of acid chlorine compounds and/or the content of hydrolyzable chlorine in the corresponding isocyanates obtained in the production process.

The invention relates to a process for producing isocyanates by a gas-phase phosgenation of the corresponding amines, wherein an amine mixture formed of at least one first amine and at least one second amine different from the first amine is reacted with phosgene to afford a reaction mixture. The invention further relates to a product obtainable or obtained by this process and to the use of such a product. The invention also relates to the use of an amine mixture formed of at least one first amine and at least one second amine different from the first amine in a process for producing the corresponding isocyanates by gas-phase phosgenation for reducing the content of acidic chlorine compounds and/or the content of hydrolyzable chlorine of the corresponding isocyanates obtained in the production process.

Isocyanates, in particular diisocyanates, in some cases also low-molecular-weight triisocyanates, as well as the higher-molecular-weight polyaddition products with terminal isocyanate groups obtainable from said compounds, are valuable raw materials for the production of polyurethanes. It is industrially advantageous to produce isocyanates by phosgenation of the parent amines either in the condensed phase (liquid-phase phosgenation, hereinafter also referred to as LPP) or in the gas phase (gas-phase phosgenation, hereinafter also referred to as GPP). The latter is practicable only in the case of amines that can be vaporized at the chosen pressure without decomposition. In general, it has been found that many isocyanates that are not producible by LPP, or producible only in poor yields or with unsatisfactory purity, can be better produced by GPP. This is true in particular of diisocyanates bearing sensitive functional groups in their molecular structure, for example ether bridges (cf. EP-A 764633 and sources cited therein).

DE 2249459 describes the phosgenation of a mixture of two chemically different amines. In this case it is essential to use an aromatic amine as the “base amine” for the production of aliphatic isocyanates from the corresponding amines, in order to avoid having to build a costly separate plant for the aliphatic isocyanates, which are usually required in significantly smaller volumes in the industry. However, the isocyanates described therein still have relatively high HC contents (0.1% and above). Moreover, in DE 2249459, a gas-phase phosgenation is not possible, on account of the different reactivities and boiling behavior/vapor pressures of the aromatic base amines relative to the aliphatic amines.

In the production of isocyanates from aliphatic and araliphatic amines, it is possible (without being bound to any particular theory) for intra- or intermolecular elimination of ammonia to occur, especially at higher temperatures such as those necessary for GPP, for example. This is illustrated schematically in the examples below.

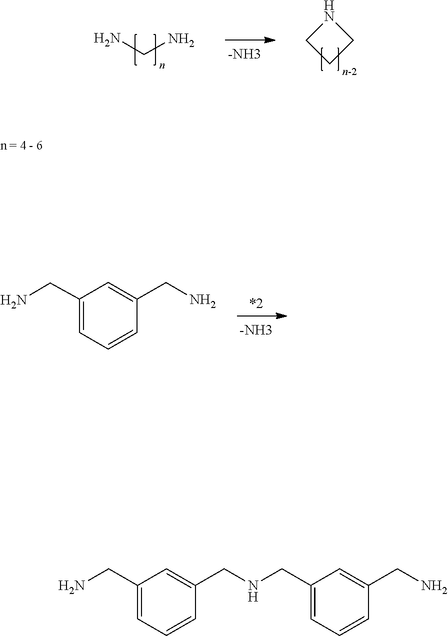

These side reactions lead to unwanted by-products that contaminate the actual products, reduce the yield, and result in deposits accompanied by increasing blockage of the phosgenation plant, since the ammonia produced in the phosgenation plant can also result in the formation of NH₄Cl, for example. This in turn ultimately not only makes it difficult to clean the plant, but can also lead to a rise in pressure in the plant equipment during operation of the plant for GPP, especially when operated for prolonged periods.

The object of the present invention was accordingly to provide a process for producing aliphatic and/or araliphatic isocyanates by gas-phase phosgenation of the corresponding amines that has improved yield and purity, in particular purity in the form of a reduction in the content of acidic chlorine compounds (AC value) and/or content of hydrolyzable chlorine (HC value). A further object of the present invention was that, as far as possible, no rise in pressure in the phosgenation plant is recorded.

This object is achieved by a process having the features of claim 1.

The invention also relates to the use of an amine mixture formed of at least one first amine and at least one second amine different from the first amine in a process for producing the corresponding isocyanates by phosgenation according to the process of the invention for reducing the content of acidic chlorine compounds and/or the content of hydrolyzable chlorine of the corresponding isocyanates obtained in the production process.

The invention further relates to a product obtainable or obtained by the process of the invention, preferably directly obtainable or obtained by the process of the invention.

In addition, the invention relates to the use of the product of the invention and/or of the isocyanate or isocyanate mixture obtained or obtainable by the process of the invention as a component for the production of polyurethanes, in particular polyurethane foams, polyurethane coatings, and polyurethane adhesives, of pharmaceutical products, in particular active substances, and also of auxiliaries, in particular auxiliaries for wet-strength finishing of paper and other cellulose products, emulsifiers, and thickeners.

Surprisingly, it was found that the yield and purity of the isocyanates (hereinafter also referred to as first isocyanate or isocyanate 1) from the corresponding aliphatic and/or araliphatic amines (hereinafter also referred to as first amine or alternatively amine 1) can be increased during phosgenation when the reaction takes place in the presence of cycloaliphatic amines (hereinafter also referred to as second amine or alternatively amine 2). The reaction of such a mixture is also referred to hereinafter as co-phosgenation, or CoPg for short. The resulting isocyanates 1 are at least equivalent in yield and purity (particularly in the content of acidic chlorine compounds (AC value) and/or of hydrolyzable chlorine compounds (HC value)) to those resulting from phosgenation of the amines 1 on their own. In addition, it was found that fewer impurities are observed with the process of the invention and that pressure rises remain constant for a longer period of time, i.e. no significant pressure rises are recorded in the given time. An extension of phosgenation run time was accordingly also observed with the process of the invention, i.e. it was possible for a phosgenation operation to be carried out in the plant for a longer time. Consequently, the process of the invention can also be used to extend the run time (increase the service life) of the phosgenation.

A measure of purity employed in the context of the invention is in particular the content of acidic chlorine compounds and the content of hydrolyzable chlorine, both the content of acidic chlorine compounds and the content of hydrolyzable chlorine being determined according to the standard ISO 15028:2014. As a further measure of the purity and general structural composition of the products of the invention, it is also possible to use gas chromatography (GC), or alternatively high-performance liquid chromatography (HPLC), in the latter case normally after derivatization of the NCO groups, with the identity of the products usually characterized by known methods (cf. also C. Six, F. Richter, Ullmann's Encyclopedia of Industrial Chemistry, “Isocyanates, Organic”, Wiley-VCH, 2003) such as mass spectrometry (GC-MS; HPLC-MS, usually in “high resolution” mode=HRMS).

The invention relates to a process for producing isocyanates by a gas-phase phosgenation of the corresponding amines, wherein an amine mixture formed of at least one first amine and at least one second amine different from the first amine is reacted with phosgene to afford a reaction mixture. The first amine is here selected from the group comprising or consisting of aliphatic and/or araliphatic amines, whereas the second amine is selected from the group comprising or consisting of cycloaliphatic amines. Preferably, the first amine and/or the second amine is a diamine.

In addition it is preferable that the aliphatic amine comprises or consists of a diaminoalkane having 4 to 15 hydrocarbon atoms, wherein it is preferable that the carbon chain from the first to the second amino group of this diaminoalkane has 4 to 6 carbon atoms (i.e. the carbon atoms between the two amino groups). Suitable diaminoalkanes are selected in particular from the group comprising or consisting of 1,4-diaminobutane, 1,4-diamino-4-methylpentane, 1,5-diaminopentane, 1,5-diamino-hexane, 1,6-diaminohexane, 2,2,4-trimethyl-1,6-diaminohexane, 2,4,4-trimethyl-1,6-diaminohexane or mixtures thereof.

The araliphatic amine is preferably selected from the group comprising or consisting of xylylene-1,3-diamine, xylylene-1,4-diamine, 1,1′-(1,2-phenylene)bis(ethan-1-amine), 1,1′-(1,3-phenylene)-bis-(ethan-1-amine), 1,1′-(1,4-phenylene)bis(ethan-1-amine), 2,2′-(1,2-phenylene)bis(propan-2-amine), 2,2′-(1,4-phenylene)bis(propan-2-amine), 2,2′-(1,3-phenylene)bis(propan-2-amine), 3-(aminomethyl) aniline, 4-(aminomethyl) aniline or mixtures thereof.

In addition, the cycloaliphatic amine is preferably selected from the group comprising or consisting of 1-amino-3,5,5-trimethyl-5-aminomethylcyclohexane; amino-[(aminocyclohexyl)methyl]-cyclohexane, especially 4,4′-methylenebis(cyclohexan-1-amine), 2-((4-aminocyclohexyl)-methyl)cyclohexan-1-amine, and 2,2′-methylenebis(cyclohexan-1-amine); 1,3-bis(aminomethyl)-cyclohexane; 1,4-bis(aminomethyl)cyclohexane; diaminocyclohexane, especially cyclohexane-1,2-diamine, cyclohexane-1,3-diamine, and cyclohexane-1,4-diamine; methyldiaminocyclohexane, especially 4-methylcyclohexane-1,3-diamine and 2-methylcyclohexane-1,3-diamine, 4,4′-methylenebis(2-methylcyclohexan-1-amine); or mixtures thereof, preferably 1-amino-3,5,5-trimethyl-5-aminomethylcyclohexane; amino-[(aminocyclohexyl)methyl]cyclohexane, especially 4,4′-methylenebis(cyclohexan-1-amine), 2-((4-aminocyclohexyl)methyl)cyclohexan-1-amine, and 2,2′-methylenebis(cyclohexan-1-amine); or mixtures thereof.

In a likewise preferred embodiment, the araliphatic amine comprises xylylene-1,3-diamine and/or xylylene-1,4-diamine, preferably the araliphatic amine is xylylene-1,3-diamine, and the cycloaliphatic amine comprises 1,3-bis(aminomethyl)cyclohexane and/or 1,4-bis(aminomethyl)-cyclohexane, preferably the cycloaliphatic amine is 1,3-bis(aminomethyl)cyclohexane.

The mass fraction of the first amine is preferably 5.0% to 80.0% by weight, more preferably 10.0% to 60.0% by weight and that of the second amine is preferably 20.0% to 95.0% by weight, more preferably 40.0% to 90.0% by weight, based on the total weight of the amine mixture.

The molar ratio of phosgene to the amino groups of the amines in the amine mixture is preferably ≥1:1 to ≤5:1, more preferably >1:1 to ≤3:1.

In addition, an inert substance is preferably used in the reaction. Suitable inert substances are selected from the group comprising or consisting of
inert gases, in particular nitrogen, argon or a mixture thereof;
inert solvents, in particular aromatic hydrocarbons, preferably chlorobenzene, o-dichlorobenzene, toluene, xylene or mixtures thereof;
or mixtures of the aforementioned inert gases and inert solvents.

The isocyanate corresponding to the first amine and/or the isocyanate corresponding to the second amine is preferably separated from the reaction mixture, separation preferably being effected by distillation, in particular thin-film distillation, extraction, crystallization, recrystallization or a combination thereof, and with the respective isocyanates obtained separately or as an isocyanate mixture.

In addition, it is preferable that the content of acidic chlorine compounds in the separated isocyanate(s) or in the isocyanate mixture is from 1 to 200 ppm, further preferably from 1 to 100 ppm, more preferably from 2 to 80 ppm, most preferably from 5 to 50 ppm, determined according to the ISO 15028:2014 standard. In particular, it is preferable that the content of acidic chlorine compounds in the separated isocyanate(s) or in the isocyanate mixture is from 1 to 200 ppm when the first amine comprises or consists of 1,4-diaminobutane (BDA) or from 1 to 100 ppm for all other amines, further preferably from 2 to 80 ppm, more preferably from 5 to 50 ppm, in each case determined according to the ISO 15028:2014 standard. It is also preferable that the content of hydrolyzable chlorine in the separated isocyanate(s) or in the isocyanate mixture is from 1 to 700 ppm, further preferably from 1 to 500 ppm, more preferably from 5 to 100 ppm, determined according to the ISO 15028:2014 standard. In particular, the content of hydrolyzable chlorine in the separated isocyanate(s) or in the isocyanate mixture is from 1 to 700 ppm when the first amine comprises or consists of 1,4-diaminobutane (BDA) or from 1 to 500 ppm for all other amines, preferably from 5 to 100 ppm, in each case determined according to the ISO 15028:2014 standard. The use of an amine mixture formed of at least one first amine and at least one second amine different from the first amine in a process for producing the corresponding isocyanates by phosgenation according to the process of the invention can accordingly be employed for reducing the content of acidic chlorine compounds and/or the content of hydrolyzable chlorine of the corresponding isocyanates obtained in the production process.

The invention further relates to a product obtainable or obtained, preferably directly obtainable or obtained, by the process of the invention.

The process products of the invention are valuable raw materials for the production of polyurethanes, adhesives, coating agents, oligomeric isocyanate modification products such as uretdione-, isocyanurate-, iminooxadiazinedione-, oxadiazinetrione-, carbodiimide-, biuret-, urethane- and allophanate-containing polyisocyanates, auxiliaries such as those used for the wet-strength finishing of paper and other cellulose products, for example, as emulsifiers, thickeners, etc., as a raw material for the production and/or formulation of active substances, pharmaceuticals, etc. The invention therefore also relates to the use of the product of the invention and/or of the isocyanate or isocyanate mixture of the invention obtained or obtainable by the process of the invention as a component for the production of polyurethanes, in particular polyurethane foams, polyurethane coatings, and polyurethane adhesives, of pharmaceutical products, in particular active substances, and also of auxiliaries, in particular auxiliaries for wet-strength finishing of paper and other cellulose products, emulsifiers, and thickeners.

The GPP process of the invention is in its main features (i.e. in respect of the plant construction, etc.) executed by means of a prior art method known per se, for example according to the teaching of EP 0 570 799 or EP 0676392 and process variants cited therein. For this purpose, the coreactants are introduced into suitable reactors close to or above the boiling temperature of the starting amine (mixture), mixed, and reacted with each other. Depending on the selected pressure, the temperatures for this are between 10° and 600° C., preferably between 15° and 500° C. The process is carried out in a pressure range of between 10 mbar and 5 bar, preferably 200 mbar and 3 bar. The reaction components in the gas-phase phosgenation can be supplied with or without the use of inert additives such as carrier gases for the phosgenation reaction. Suitable carrier gases include nitrogen, argon or other inert gases, as well as vapors of technically available solvents, such as chlorobenzene, dichlorobenzene, xylenes, chloronaphthalenes, decahydronaphthalene, etc.

The isocyanate mixtures of the invention are subsequently obtained by cooling the gas stream down to a temperature above the decomposition temperature of the corresponding carbamoyl chloride intermediates. In a preferred embodiment, the isocyanate corresponding to the first amine and/or the isocyanate corresponding to the second amine is then separated from the reaction mixture, separation preferably being effected by distillation, in particular thin-film distillation, extraction, crystallization, recrystallization or a combination thereof, and with the respective isocyanates obtained separately or as an isocyanate mixture. Particularly preferably, the isocyanate corresponding to the first amine and the isocyanate corresponding to the second amine are separated from the reaction mixture. It is also preferable that separation by distillation is optionally followed by recrystallization.

### EMBODIMENTS

The present invention relates in particular to the following embodiments:

In a first embodiment, the invention relates to a process for producing isocyanates by a gas-phase phosgenation of the corresponding amines, wherein an amine mixture formed of at least one first amine and at least one second amine different from the first amine is reacted with phosgene to afford a reaction mixture, characterized in that
the first amine is selected from the group comprising or consisting of aliphatic and/or araliphatic amines; and
the second amine is selected from the group comprising or consisting of cycloaliphatic amines.

In a second embodiment, the invention relates to a process according to embodiment 1, characterized in that the first amine and/or the second amine is a diamine.

In a third embodiment, the invention relates to a process according to embodiment 1 or 2, characterized in that the aliphatic amine comprises or consists of a diaminoalkane having 4 to 15 hydrocarbon atoms, wherein it is preferable that the carbon chain from the first to the second amino group of this diaminoalkane has 4 to 6 carbon atoms and/or that this diaminoalkane is selected from the group comprising or consisting of 1,4-diaminobutane, 1,4-diamino-4-methylpentane, 1,5-diaminopentane, 1,5-diaminohexane, 1,6-diaminohexane, 2,2,4-trimethyl-1,6-diaminohexane, 2,4,4-trimethyl-1,6-diaminohexane or mixtures thereof.

In a fourth embodiment, the invention relates to a process according to any of the preceding embodiments, characterized in that the araliphatic amine is selected from the group comprising or consisting of xylylene-1,3-diamine, xylylene-1,4-diamine, 1,1′-(1,2-phenylene)bis(ethan-1-amine), 1,1′-(1,3-phenylene)bis(ethan-1-amine), 1,1′-(1,4-phenylene)bis(ethan-1-amine), 2,2′-(1,2-phenylene)bis(propan-2-amine), 2,2′-(1,4-phenylene)bis(propan-2-amine), 2,2′-(1,3-phenylene)bis(propan-2-amine), 3-(aminomethyl)aniline, 4-(aminomethyl)aniline or mixtures thereof.

In a fifth embodiment, the invention relates to a process according to any of the preceding embodiments, characterized in that the cycloaliphatic amine is selected from the group comprising or consisting of 1-amino-3,5,5-trimethyl-5-aminomethylcyclohexane; amino-[(aminocyclohexyl)methyl]cyclohexane, especially 4,4′-methylenebis(cyclohexan-1-amine), 2-((4-aminocyclohexyl)methyl)cyclohexan-1-amine, and 2,2′-methylenebis(cyclohexan-1-amine); 1,3-bis(aminomethyl)cyclohexane; 1,4-bis(aminomethyl)cyclohexane; diaminocyclohexane, especially cyclohexane-1,2-diamine, cyclohexane-1,3-diamine, and cyclohexane-1,4-diamine; methyldiaminocyclohexane, especially 4-methylcyclohexane-1,3-diamine and 2-methylcyclohexane-1,3-diamine, 4,4′-methylenebis(2-methylcyclohexan-1-amine); or mixtures thereof, preferably 1-amino-3,5,5-trimethyl-5-aminomethylcyclohexane; amino-[(aminocyclohexyl)methyl]cyclohexane, especially 4,4′-methylenebis(cyclohexan-1-amine), 2-((4-aminocyclohexyl)methyl)cyclohexan-1-amine, and 2,2′-methylenebis(cyclohexan-1-amine); or mixtures thereof.

In a sixth embodiment, the invention relates to a process according to any of the preceding embodiments, characterized in that the mass fraction
of the first amine is 5.0% to 80.0% by weight, preferably 10.0% to 60.0% by weight and
of the second amine is 20.0% to 95.0% by weight, preferably 40.0% to 90.0% by weight, based on the total weight of the amine mixture.

In a seventh embodiment, the invention relates to a process according to any of the preceding embodiments, characterized in that the molar ratio of phosgene to the amino groups of the amines in the amine mixture is ≥1:1 to ≤5:1, preferably >1:1 to ≤3:1.

In an eighth embodiment, the invention relates to a process according to any of the preceding embodiments, characterized in that an inert substance is employed in the reaction, the inert substance being selected from the group comprising or consisting of:
inert gases, in particular nitrogen, argon or a mixture thereof;
inert solvents, in particular aromatic hydrocarbons, preferably chlorobenzene, o-dichlorobenzene, toluene, xylene or mixtures thereof;
or mixtures of the aforementioned inert gases and inert solvents.

In a ninth embodiment, the invention relates to a process according to any of the preceding embodiments, characterized in that the isocyanate corresponding to the first amine and/or the isocyanate corresponding to the second amine is separated from the reaction mixture, separation preferably being effected by distillation, in particular thin-film distillation, extraction, crystallization, recrystallization or a combination thereof, and with the respective isocyanates obtained separately or as an isocyanate mixture.

In a tenth embodiment, the invention relates to a process according to any of embodiments 1 to 8, characterized in that the isocyanate corresponding to the first amine and/or the isocyanate corresponding to the second amine is separated from the reaction mixture, separation preferably being effected by distillation, in particular thin-film distillation, extraction, crystallization, recrystallization or a combination thereof, and with the respective isocyanates obtained as an isocyanate mixture.

In an eleventh embodiment, the invention relates to a process according to embodiment 9 or 10, characterized in that the content of acidic chlorine compounds in the separated isocyanate(s) or in the isocyanate mixture is from 1 to 200 ppm, preferably from 1 to 100 ppm, further preferably from 2 to 80 ppm, more preferably from 5 to 50 ppm, determined according to the ISO 15028:2014 standard.

In a twelfth embodiment, the invention relates to a process according to embodiment 9 to 11, characterized in that the content of hydrolyzable chlorine in the separated isocyanate(s) or in the isocyanate mixture is from 1 to 700 ppm, preferably from 1 to 500 ppm, more preferably from 5 to 100 ppm, determined according to the ISO 15028:2014 standard.

In a thirteenth embodiment, the invention relates to the use of an amine mixture formed of at least one first amine and at least one second amine different from the first amine in a process for producing the corresponding isocyanates by gas-phase phosgenation according to a process according to any of embodiments 1 to 12 for reducing the content of acidic chlorine compounds and/or the content of hydrolyzable chlorine of the corresponding isocyanates obtained in the production process.

In a fourteenth embodiment, the invention relates to a product obtainable or obtained by a process according to any of embodiments 1 to 12, preferably according to any of embodiments 1 to 8 and 10 to 12, preferably directly obtainable or obtained by a process according to any of embodiments 1 to 12, preferably according to any of embodiments 1 to 8 and 10 to 12.

In a fifteenth embodiment, the invention relates to the use of the product according to embodiment 14 and/or of the isocyanate or isocyanate mixture obtained or obtainable by a process according to any of embodiments 9 to 12 as a component for the production of polyurethanes, in particular polyurethane foams, polyurethane coatings, and polyurethane adhesives, of pharmaceutical products, in particular active substances, and also of auxiliaries, in particular auxiliaries for wet-strength finishing of paper and other cellulose products, emulsifiers, and thickeners.

### EXAMPLES

The examples described below are intended to describe the process and some of the process products of the invention more particularly without limiting the invention; all percentages are percentages by weight unless otherwise stated.

The content of acidic chlorine compounds and the content of hydrolyzable chlorine was determined in accordance with the ISO 15028:2014 standard.

GC-MS was carried out using the Agilent GC6890 equipped with an MN 725825.30 Optima-5 MS Accent capillary column (30 m, 0.25 mm internal diameter, 0.5 μm film layer thickness) and a 5973 mass spectrometer as detector with helium as transport gas (flow rate of 2 ml/min). The column temperature was initially 60° C. (2 min) and was then gradually increased to 360° C. at 8 K/min. The GC-MS detection employed electron-impact ionization with 70 eV ionization energy. An injector temperature of 250° C. was chosen.

In some cases, NMR spectroscopic investigations were employed for further structural elucidation. The measurements were carried out on a Bruker DRX 700 instrument on approx. 1% (1H NMR) or approx. 50% (¹³C NMR) samples in dry C₆D₆ at a measurement frequency of 700 MHZ (1H NMR) or 176 MHz (¹³C NMR). The C₆D₅H present in the solvent was used as reference signal for the ppm scale: ¹H-NMR chemical shift 7.15 ppm, ¹³C-NMR chemical shift 128.02 ppm.

All reactions were carried out under a nitrogen atmosphere in glass apparatus dried beforehand under reduced pressure at 150-200° C.

All chemicals used in the examples according to the invention and in the comparative examples were obtained from Aldrich, D-82018 Taufkirchen. Hereinafter BDA stands for 1,4-diaminobutane, PDA for 1,5-diaminopentane, HDA for 1,6-diaminohexane, XDA for 1,3-xylenediamine, and MDA for 4,4′-methylenedianiline.

### General Procedures

Procedure A—Liquid-Phase Phosgenations (LPP) in the Presence of MDA (Comparison with DE 2249459):

A 4-necked flask cooled to −5° C. with mechanical stirrer, dropping funnel, internal thermometer, and gas inlet tube was initially charged with 500 ml of monochlorobenzene (MCB) and then twice the molar amount of phosgene based on diamine (mixture) to be reacted was condensed in and the solution of the respective amine (mixture) was slowly added dropwise to the MCB, with further cooling at −10 to max. 0° C. After all the amine (mixture) had been added, the dropping funnel was filled with 100 ml of MCB and this was likewise added dropwise to the reaction mixture. While introducing phosgene (approx. 120 g/h) via the gas-inlet tube, the temperature was then gradually increased until MCB reflux occurred. On reaching the boiling temperature, stirring was continued under reflux with further passage of phosgene through the mixture (approx. 120 g/h) until a clear solution had formed, but for at least 5 hours. For dephosgenation, stirring was continued under reflux but passing dry nitrogen instead of phosgene through the mixture via the gas-inlet tube until absence of phosgene was ensured by phosgene indicator paper. The further workup is described in the respective examples.

### Procedure B—Gas-Phase Phosgenations (GPP):

In a glass system having a heated mixing tube (reactor temperature=T1) 2.5 mm in diameter and 17.5 mm in length with a downstream condensation stage and subsequent phosgene adsorption tower filled with activated carbon, phosgene that had been preheated in an upstream heat exchanger (temperature=T2) was continuously introduced through a nozzle projecting into the mixing tube. This was accompanied by the simultaneous introduction into the reaction space, through the annular gap between the nozzle and the mixing tube, of a likewise preheated amine mixture (temperature=T3) at a metering rate optimized according to the particular amine (mixture), which was optionally diluted with dry nitrogen as diluent that was likewise preheated to T3. The application of a reduced pressure at the end of the condensation stage allowed a defined pressure to be maintained in the mixing tube. The hot reaction mixture exiting the reaction space in gaseous form was conducted into a condensation stage by 1,2-dichlorobenzene boiling under reflux. This brought about the selective condensation of the diisocyanates that had formed. The gas mixture passing through the scrubbing stage, which consisted essentially of nitrogen, HCl, and excess phosgene, was subsequently freed of phosgene in the adsorption tower and the condensate freed of residual, dissolved phosgene as described under A. The further workup is described in the respective examples.

### Comparative Examples 1—Liquid-Phase Phosgenation of Sensitive Aliphatic and Araliphatic Diamines in the Presence of MDA (Comparison with DE 2249459)

In accordance with general procedure A, a mixture of 0.04 mol of amine 1 and 31.8 g of MDA was in each case subjected to liquid-phase phosgenation, which afforded, after distillative workup, the diisocyanates corresponding to the amines 1 in the listed purity and yield. The distillation residue (MDI) was not investigated in detail.

TABLE 1
Results for comparative examples 1

| | | Yield¹⁾ | Purity²⁾ | AC | HC |
|---|---|---|---|---|---|
| Amine 1 | Amine 2 | Isocyanate 1 | Isocyanate 1 | [ppm] | [ppm] |
| BDA | MDA | 85.4 | 96.4 | 7128 | 7403 |
| PDA | MDA | 92.1 | 97.5 | 842 | 922 |
| HDA | MDA | 95.8 | 98.3 | 256 | 289 |
| XDA | MDA | 88.4 | 95.5 | 1029 | 1288 |

¹⁾[% of theor. based on amine 1];
²⁾Acc. to GC, area %, 100% method without factorization

All diisocyanates are contaminated with chlorine-containing secondary components and are unusable for employment in typical polyurethane applications.

### Comparative Examples 2—Gas-Phase Phosgenation of Sensitive Aliphatic and Araliphatic Diamines

In accordance with general procedure B, the amines listed in Table 2 were subjected to gas-phase phosgenation under the conditions listed in Table 2.

TABLE 2
Results for comparative examples 2

| | | | AC | HC |
|---|---|---|---|---|
| Amine | Yield¹⁾ | Purity²⁾ | [ppm] | [ppm] |
| BDA | 98.4 | 98.2 | 768 | 956 |
| PDA | 98.5 | 98.8 | 532 | 622 |
| HDA | 98.9 | 99.1 | 155 | 196 |
| XDA | 92.4 | 96.4 | 856 | 955 |

¹⁾[% of theor. based on amine metered in];
²⁾Acc. to GC, area %, 100% method without factorization

After every hour of metering time, the condensate obtained (1,2-dichlorobenzene, diisocyanate, by-products) was isolated, dephosgenated separately, analyzed, and the respective experiment continued under otherwise unchanged conditions. The dephosgenated crude products thus obtained were then combined and worked up by distillation. Table 2 gives details of the collective yield and purity of the diisocyanates obtained. In all cases, the diisocyanates obtained showed a steady increase in contamination by undesirable secondary components over the course of the experiment. In the case of XDA/XDI, the experiment had to be abandoned after approx. 2 h, since persistent caking and deposits had formed in the reaction space, which led to a significant rise in pressure.

### Example 3 (According to the Invention)—CoPg-GPP of XDA (Amine 1) and PACM 20 (Amine 2) By GPP According to General Procedure B

In accordance with general procedure B, mixtures of XDA (amine 1) and PACM 20 (amine 2) were phosgenated at a uniform 450° C. (T1=T2=T3). In this case, at the start of the experiment pure amine 2 (˜0.330 mol/h) was metered in with a molar phosgene excess of about 250%. After an experiment time of 30 minutes, this was supplemented with amine 1 such that an amine mixture consisting of 25 mol % of amine 1 and 75 mol % of amine 2 was being metered in, cf. Table 3, example 3a. The concentration of amine 1 was then increased further (example 3b), such that an amine mixture with 50 mol % of amine 1/50 mol % of amine 2 was used. In total, 113.0 g (0.83 mol) of XDA and 383.3 g (1.82 mol) of PACM 20 were processed. The amounts metered in, phosgene excesses used, and run times are given in Table 3. No rise in pressure in the system was recorded.

TABLE 3
GPP of XDA and PACM 20

| | XDA | PACM | Amount of | Phosgene | | Run | Reactor |
|---|---|---|---|---|---|---|---|
| | [mol- | 20 | amine | excess | Pressure | time | deposits |
| Example | %] | [mol-%] | [mol/h] | [mol-%] | [mbar] | [h] | [g] |
| 3a | 25 | 75 | 0.30 | 276 | 500 | 4.38 | 1.2 |
| 3b | 50 | 50 | 0.33 | 242 | 500 | 4 | 1.3 |

The dephosgenated crude products were investigated by ¹H-NMR spectroscopy and GC-MS and then combined, since there were no differences in product purity based on XDI.

Distillative workup through a 40 cm column packed with #1 Interpack random packing (internal diameter approx. 25 mm) gave only 22.4 g of a dark brown, highly viscous distillation residue and 148.2 g of XDI and also 463.8 g of H₁₂-MDI, in each case as light-colored liquids that according to a combination of analytical methods (NMR, GC-MS) consisted to an extent of >99% of XDI or H₁₂-MDI (isomer mixture) (94.9% and 97.2% yield respectively based on the amines used). The AC/HC content was 35/120 ppm (XDI) and 27/38 ppm (H₁₂-MDI). Discoloration was not observed even after weeks of storage with exclusion of air.

### Example 4 (According to the Invention)—CoPg-GPP of BDA (Amine 1) and IPDA (Amine 2) by GPP According to General Procedure B

In accordance with general procedure B, a mixture consisting of 33.7 g of BDA (amine 1) and 281.4 g of IPDA (amine 2) was phosgenated at a uniform 350° C. (T1=T2=T3) and 500 mbar system pressure. No rise in pressure in the system during the experiment was recorded. After separation of the residue, which left behind 10.4 g of a dark brown, highly viscous distillation residue, the dephosgenated crude product was distilled through a 40 cm column packed with #1 Interpack random packing (internal diameter approx. 25 mm). 52.8 g of BDI having a purity of 98.8% (GC) was obtained. The AC/HC content was 117/649 ppm. A second fraction of 362.8 g of IPDI having a purity of 99.2% (GC), AC/HC: 122/165 ppm, was obtained.

### Example 5 (According to the Invention)—CoPg-GPP of PDA (Amine 1, Example 5a) and HDA (Amine 1, Example 5b) with PACM 20 (Amine 2) by GPP According to General Procedure B

In accordance with general procedure B, a mixture consisting of 39.1 g of PDA as amine 1 and 348 g PACM 20 as amine 2 (example 5a) or 44.4 g of HDA as amine 1 and 348 g of PACM 20 as amine 2 (example 5b) was phosgenated at a uniform 450° C. (T1=T2=T3) and 500 mbar system pressure. No rise in pressure in the system during the experiment was recorded. After separation of the residue, which left behind respectively 3.4 g (example 5a) and 2.9 g (example 5b) of a dark brown, highly viscous distillation residue, the dephosgenated crude product was distilled through a 40 cm column packed with #1 Interpack random packing (internal diameter approx. 25 mm). This afforded in example 5a 54.8 g of PDI having a purity of 99.2% (GC). The AC/HC content was 38/87 ppm and in example 5b 58.9 g of HDI having a purity of 99.8% (GC) was obtained. The AC/HC content was 22/42 ppm. The diisocyanate 2 left behind as a high boiler was not investigated in detail.

A comparison of the GC-MS results obtained for the diisocyanates 1, PDI, and HDI obtained according to the invention with the data obtained for the corresponding compounds from comparative examples 1 and 2 shows that the concentration of all the secondary components in the diisocyanates obtained according to the invention is significantly lower than in the diisocyanates obtained by known prior art methods. In the case of PDI, this concerns in particular chlorine-containing compounds having molecular weights (from HRMS) of 145.03, 147.05, 178.99, and 208.02 (the latter each having isotope patterns indicating the presence of at least 2 Cl atoms in the respective molecules), some of these compounds evidently having structural isomers, as evidenced by GC-HRMS signals with retention times very close to one another and very similar fragmentation patterns in the mass spectra. In the case of HDI, the number of Cl-containing secondary components is significantly lower than with PDI and is essentially limited to the compounds known from the literature: tetrahydro-1H-azepine-1-carbonyl chloride (several isomers), azepane-1-carbonyl chloride and (in traces) chlorotetrahydro-1H-azepine-1-carbonyl chloride (2 Cl atoms, several isomers).

The invention claimed is:

1. A process for producing isocyanates by a gas-phase phosgenation of amines, wherein an amine mixture formed of at least one first amine and at least one second amine different from the first amine is reacted with phosgene to provide a reaction mixture,
wherein
the first amine comprise aliphatic or araliphatic amines, and
the second amine comprise cycloaliphatic amines.

2. The process claim 1, wherein the first amine or the second amine is a diamine.

3. The process claim 1, wherein the aliphatic amine comprises diaminoalkane having 4 to 15 hydrocarbon atoms.

4. The process of claim 1, wherein the araliphatic amine is xylylene-1,3-diamine, xylylene-1,4-diamine, 1,1′-(1,2-phenylene)bis(ethan-1-amine), 1,1′-(1,3-phenylene)bis(ethan-1-amine), 1,1′-(1,4-phenylene)bis(ethan-1-amine), 2,2′-(1,2-phenylene)bis(propan-2-amine), 2,2′-(1,4-phenylene)bis(propan-2-amine), 2,2′-(1,3-phenylene)bis(propan-2-amine), 3-(aminomethyl) aniline, 4-(aminomethyl) aniline or mixtures thereof.

5. The process of claim 1, wherein the cycloaliphatic amine is 1-amino-3,5,5-trimethyl-5-aminomethylcyclohexane, amino-[(aminocyclohexyl)methyl]-cyclohexane, 4,4′-methylenebis(cyclohexan-1-amine), 2-((4-aminocyclohexyl)methyl)cyclohexan-1-amine, 2,2′-methylenebis(cyclohexan-1-amine), 1,3-bis(aminomethyl)cyclohexane, 1,4-bis(amnomethyl)cyclohexane, diaminocyclohexane, cyclohexane-1,2-diamine, cyclohexane-1,3-diamine, and cyclohexane-1,4-diamine, methyldiaminocyclohexane, 4-methylcyclohexane-1,3-diamine, 2-methylcyclohexane-1,3-diamine, 4,4′-methylenebis(2-methylcyclohexan-1-amine), or mixtures thereof.

6. The process of claim 1, wherein the mass fraction
of the first amine is 5.0% to 80.0% by weight, % by and
the mass fraction of the second amine is 20.0% to 95.0% by weight
based on the total weight of the amine mixture.

7. The process of claim 1, wherein the molar ratio of phosgene to the amino groups of the amines in the amine mixture is ≥1:1 to ≤5:1.

8. The process of claim 1, wherein an inert substance is employed in the reaction, the inert substance being
inert gases,
inert solvents,
or mixtures thereof.

9. The process of claim 1, further comprising separating the isocyanates produced from the first amine or the isocyanates produced from the second amine, from the reaction mixture, and with the isocyanates are obtained separately or as an isocyanate mixture.

10. The process of claim 1, further comprising separating the isocyanates produced from the first amine or the isocyanates produced from the second amine, from the reaction mixture, wherein the separating is done by distillation, extraction, crystallization, recrystallization or a combination thereof, and the isocyanates are obtained as an isocyanate mixture.

11. The process of claim 9, wherein the content of acidic chlorine compounds in the separated isocyanate or in the isocyanate mixture is from 1 to 200 ppm, determined according to the ISO 15028:2014 standard.

12. The process of claim 9, wherein the content of hydrolyzable chlorine in the separated isocyanate or in the isocyanate mixture is from 1 to 700 ppm, determined according to the ISO 15028:2014 standard.

13-14. (canceled)

15. A polyurethane foams, polyurethane, or adhesives comprising a polyurethane produced from the isocyanate produced by the process of claim 1.

16. The process of claim 1, wherein the aliphatic amine comprises a diaminoalkane having a carbon chain from the first to the second amino group of the diaminoalkane has 4 to 6 carbon atoms.

17. The process of claim 1, wherein the aliphatic amine comprises a diaminoalkane, wherein the diaminoalkane is 1,4-diaminobutane, 1,4-diamino-4-methylpentane, 1,5-diaminopentane, 1,5-diaminohexane, 1,6-diaminohexane, 2,2,4-trimethyl-1,6-diaminohexane, 2,4,4-trimethyl-1,6-diaminohexane or mixtures thereof.

18. The process of claim 1, wherein the mass fraction of the first amine is 10.0% to 60.0% by weight and the mass fraction of the second amine is 40.0% to 90.0% by weight, based on the total weight of the amine mixture.

19. The process of claim 1, wherein the molar ratio of phosgene to the amino groups of the amines in the amine mixture is >1:1 to ≤3:1.

20. The process of claim 9, wherein the content of acidic chlorine compounds in the separated isocyanates or in the isocyanate mixture is from 2 to 80 ppm, determined according to the ISO 15028:2014 standard.

21. The process of claim 9, wherein the content of acidic chlorine compounds in the separated isocyanates or in the isocyanate mixture is from 5 to 50 ppm, determined according to the ISO 15028:2014 standard.

22. The process of claim 9, wherein the content of hydrolyzable chlorine in the separated isocyanates or in the isocyanate mixture is from 5 to 100 ppm, determined according to the ISO 15028:2014 standard.
